(19) United States
(12) Patent Application Publication
(10) Pub. No.: US 2015/0144133 A1
(43) Pub. Date: May 28, 2015

(54) TREATMENT OF CHRONIC MIGRAINES BY OXYGEN INHALATION

(71) Applicant: Air Liquide Sante (International), Paris (FR)

(72) Inventor: Eytan ELLENBERG, Paris (FR)

(21) Appl. No.: 14/542,703

(22) Filed: Nov. 17, 2014

(30) Foreign Application Priority Data
Nov. 26, 2013 (FR) 1361628

(51) Int. Cl. A61M 16/10; A61M 16/20; A61M 16/00; A61M 16/06

(52) U.S. Cl. CPC A61M 16/101; A61M 16/0622; A61M 16/205; A61M 16/0078; A61M 2202/0208
USPC 128/202.26

(57) ABSTRACT

The invention relates to an inhalable gaseous composition containing oxygen (O₂) in a proportion greater than 50% by volume, intended to treat, to minimize or to prevent a chronic migraine attack in a patient, said composition being produced on site, i.e. at the patient's home, by an oxygen-producing device designed so as to produce a gas stream containing more than 50% of oxygen from air.

### CROSS REFERENCE TO RELATED APPLICATIONS

This application claims the benefit of priority under 35 U.S.C. §119 (a) and (b) to French Patent Application No. 1361628, filed Nov. 26, 2013, the entire contents of which are incorporated herein by reference.

### BACKGROUND

The invention relates to an oxygen-rich gaseous composition produced on site, i.e. at the patient's home, and used for the management of chronic migraine attacks in patients at their home.

Chronic migraine is a frequent complication of conventional migraines, i.e. of conventional cephalalgia or headaches.

A chronic migraine is characterized by the occurrence of more than 15 cephalalgia attacks per month for at least 3 months with at least 8 days of migraine in total, according to the Société Francaise d'Etude des Migraines et des Céphalées [French Society for the Study of Migraines and Cephalalgia].

In order to attempt to soothe their chronic migraines, some patients suffering from chronic migraines have a tendency to increase the amount and/or the doses of drugs that they are taking, namely typically tablets of nonsteroidal anti-inflammatory (NSAID) type which decrease the inflammation of meningeal vessels, or of tryptan type, which are vasoconstrictors that reduce the diameter of dilated blood vessels.

However, the increase in the amount and/or in the doses of drugs absorbed causes a high risk of drug abuse in these patients and consequences on the health of these patients, in particular in terms of side effects, of drug interactions, of addiction, etc.

Conversely, some patients are frightened by the risk of drug abuse and attempt to reduce the doses of antimigraine drugs, which then occurs to the detriment of relief of their chronic migraine.

Moreover, some patients cannot be treated with these tablets owing to their side effects. Thus, drugs of NSAID type are contraindicated in patients with a history of gastroduodenal ulcer and advised against in those with a history of kidney diseases. In an analogous manner, tryptans are contraindicated in patients with a history of cardiovascular problems, such as infarction, arterial hypertension, stroke, etc.

In addition, the negative social impact of chronic migraines is very significant since it causes in particular repeated work interruptions, increased solitude of individuals, who have a tendency to isolate themselves, etc.

Moreover, document FR-A-2929513 proposes the use of oxygen gas for treating migraines without aura in pregnant women. The oxygen used comes from gas bottles. This solution is not practical since it requires the setting up of gas bottle management logistics and is not therefore easily applicable to the treatment of patients at their home.

The problem which then arises is that of providing an effective composition, without side effects and simple to administer, which makes it possible to treat, i.e. to at least reduce, the pain caused by cephalalgia in patients suffering from chronic migraines, and which, moreover, enables patients to rapidly treat themselves as soon as their attack occurs and without needing to go to a treatment centre, such as a physician, a hospital, or the like.

### SUMMARY

The solution of the invention relates to an inhalable gaseous composition, i.e. an inhalable gaseous medicament, containing oxygen (O₂) in a proportion greater than 50% by volume, intended to treat, to minimize or to prevent a state of chronic migraine in a human being, said gaseous composition being produced on site, i.e. at the patient's home, by an oxygen-producing device designed so as to produce a gas stream containing more than 50% by volume of oxygen from air.

In the context of the present invention, the management of the migraine attacks is therefore carried out directly at home, which represents the ideal place for relieving the attacks since it offers patients a known environment, with the possibility of isolation and reduction of sound, of light or of other stressing or disturbing factors.

In the context of the present invention:
the gaseous composition preferentially makes it possible to treat or minimize a migraine attack;
the gas percentages (%) are percentages by volume;
“the chronic migraine” is defined as attacks of cephalalgia (headaches) which occur more than 15 times per month for at least 3 months with at least 8 days of migraine in total;
“gaseous composition produced on site” means that the gaseous composition is manufactured directly on the site where it is used, mainly at home, therefore on the site where it is administered to the patient by inhalation, as opposed to a medicament which is manufactured in a factory or elsewhere, and then conveyed to the site where it is used in a container, such as a gas bottle for example.

As appropriate, the inhalable gaseous composition of the invention may comprise one or more of the following technical characteristics:
it contains a proportion of oxygen greater than 60%;
it contains a proportion of oxygen greater than 70%;
it contains a proportion of oxygen greater than 80%;
it typically contains a proportion of oxygen from 85% to 96%;
it is obtained by separation of air by means of an oxygen-producing device chosen from oxygen concentrators;
it is obtained by separation of air by means of at least one adsorbent material arranged in the oxygen-producing device;
it is obtained by preferential adsorption of nitrogen on at least one adsorbent material and recovery of an oxygen-enriched gas stream;
at least one adsorbent material comprises a zeolite, preferably a zeolite at least partially exchanged with lithium, calcium, etc., cations;
the adsorbent material advantageously comprises a zeolite which is at least 80% exchanged with lithium (Li) cations, preferably between 88% and 98% exchanged with lithium cations, the residual cations being essentially sodium (Na);
it is obtained by adsorption according to production cycles of PSA (pressure swing adsorption) or VSA (vacuum swing adsorption) type, or a combination of such cycles;
it is administered during the migraine attack;
it is administered as quickly as possible after the beginning of the attack;
the administration of the gaseous composition is continued until total or partial disappearance of the migraine symptoms;
the duration of inhalation is chosen so as to result in at least a decrease in the migraine pain;
the duration of inhalation is chosen so as to result in complete disappearance of the migraine pain;
the gaseous composition inhalation time is at least 10 minutes, preferably about 15 minutes to 3 hours;
the administration of the gaseous composition is carried out continuously or batchwise, preferably continuously;
the administration of the gaseous composition is carried out continuously at a flow rate of 1 l/min to 30 l/min, preferably between 3 and 15 l/min, more preferably from 5 to 12 l/min, typically about 6 to 10 l/min;
the administration of the gaseous composition is carried out by means of a patient interface, preferably a patient interface chosen from respiratory masks and nasal canulas;
the administration of the gaseous composition is carried out by means of a “high concentration” respiratory mask comprising a mask body comprising an inhalation chamber in fluidic communication with a flexible reservoir containing at least one part of the gaseous composition;
the administration of the gaseous composition is carried out by means of a respiratory mask comprising a non-return valve placed between the inhalation chamber and the flexible reservoir;
the human being is a man or a woman, including children;
the gaseous composition is manufactured at the patient's home, from ambient air;
the gaseous composition is manufactured at the patient's home and immediately before or at the moment it is inhaled by said patient.

Moreover, the invention also relates to a method for manufacturing an inhalable gaseous composition, i.e. an inhalable medicament, containing oxygen in a proportion greater than 50% by volume, intended to treat, to minimize or to prevent a chronic migraine attack in a human being, in which said gaseous composition is produced on site, i.e. at the patient's home, by an oxygen-producing device designed so as to produce a gas stream containing more than 50% by volume of oxygen from air, in particular an oxygen concentrator as described above.

In addition, the invention also relates to a therapeutic treatment method for treating, minimizing or preventing a state of chronic migraine in an individual, namely a man or a woman suffering from chronic migraines, in which:

i) an inhalable gaseous composition, i.e. an inhalable gaseous medicament, containing more than 50% of oxygen, typically more than 80% of oxygen, is produced on site, i.e. directly on the site where it is used, by means of an oxygen-producing device designed so as to produce a gas stream containing more than 50% of oxygen from air; and

ii) said gaseous composition containing oxygen in a proportion greater than 50% by volume, obtained in step i), is administered to said individual by inhalation.

As appropriate, the method of the invention may comprise one or more of the following technical characteristics:
the oxygen-producing device is an oxygen concentrator, in particular as described above;
the oxygen-producing device produces and delivers a continuous or virtually continuous stream of said gaseous composition;
the administration of said gaseous composition is carried out by continuous inhalation of the gaseous medicament;
the administration of said gaseous composition is performed at the time of the migraine attacks, preferably until the migraine pain has disappeared;
the administration of said gaseous composition is performed for a period of 15 min to 1 h 30 min, typically for less than 1 h;
the administration of said gaseous composition is performed at a flow rate of 3 to 15 l/min;
said gaseous composition contains from 85% to 96% of oxygen.

Generally, the fact that said oxygen-rich gaseous composition, i.e. oxygen-rich gaseous medicament, is, in the context of the present invention, produced directly on site (in situ) by separation of ambient air by means of an oxygen-producing device, such as an oxygen concentrator, is particularly advantageous because:

this avoids laborious transportation logistics,
this minimizes supply interruptions and therefore the risks to the patient, in particular the risks of the latter being short of therapeutic gas,
it makes it possible to correspond as closely as possible to the ideal place chosen by patients in the event of a severe attack (home),
it provides a lower cost for health authorities, compared with the delivery of bottles in large quantities to the patient or the patient's pharmacist.

### BRIEF DESCRIPTION OF THE DRAWINGS

The present invention will now be described in greater detail with reference to the appended figures among which:

FIG. 1 represents a diagrammatic view of an oxygen-producing device A designed so as to produce, in situ, a gaseous composition according to the present invention, from ambient air; and

FIG. 2 represents diagrammatically (side view) a “high concentration” mask which can be used for administering the gaseous composition according to the present invention.

### DESCRIPTION OF PREFERRED EMBODIMENTS

More specifically, the oxygen-producing device 1 is an oxygen concentrator which makes it possible to produce the gaseous composition acting as gaseous medicament according to the invention on site, i.e. directly at the patient's home, by separation of ambient air (O₂ content approximately 21 vol. %) by preferential adsorption of nitrogen on one (or more) adsorbent material(s) and recovery of an oxygen-enriched gas stream, i.e. containing more than 50% by volume of oxygen, in particular more than 80 vol. % of oxygen, typically from 85% to 96% of oxygen, the rest being essentially nitrogen and other impurities, of the argon type, in minor amounts.

Advantageously, the adsorbent material is formed from zeolite particles or beads exchanged with metal cations. Preferentially, the zeolite is more than 80% exchanged with lithium cations. Typically, use is made of a zeolite between 88% and 98% exchanged with Li cations, the residual cations being mainly sodium and/or optionally potassium cations. Such a lithium-exchanged zeolite and the method of air separation and of oxygen production using it are described in particular by EP-A-0297542.

The particles of adsorbent material are arranged in one or more beds, preferably several beds, typically 2 or 3 beds, arranged in parallel and operating in alternating fashion. The nitrogen adsorption and the production of the oxygen stream used as inhalable gaseous composition according to the invention are then carried out via cycles of PSA or VSA type comprising one or more production phases and/or one or more regeneration phases performed at different pressures.

Once it has been produced in the oxygen concentrator 1, the gaseous stream constituting the gaseous composition according to the invention is conveyed, via a flexible pipe 18, to a patient interface 10, such as a respiratory mask, which makes it possible to administer it by continuous or fractional inhalation, to a human being 2, whether this is a man or a woman, and whatever his or her age, suffering from chronic migraines characterized by attacks of cephalalgia (headaches) occurring more than 15 times per month for at least 3 months with at least 8 days of migraine in total.

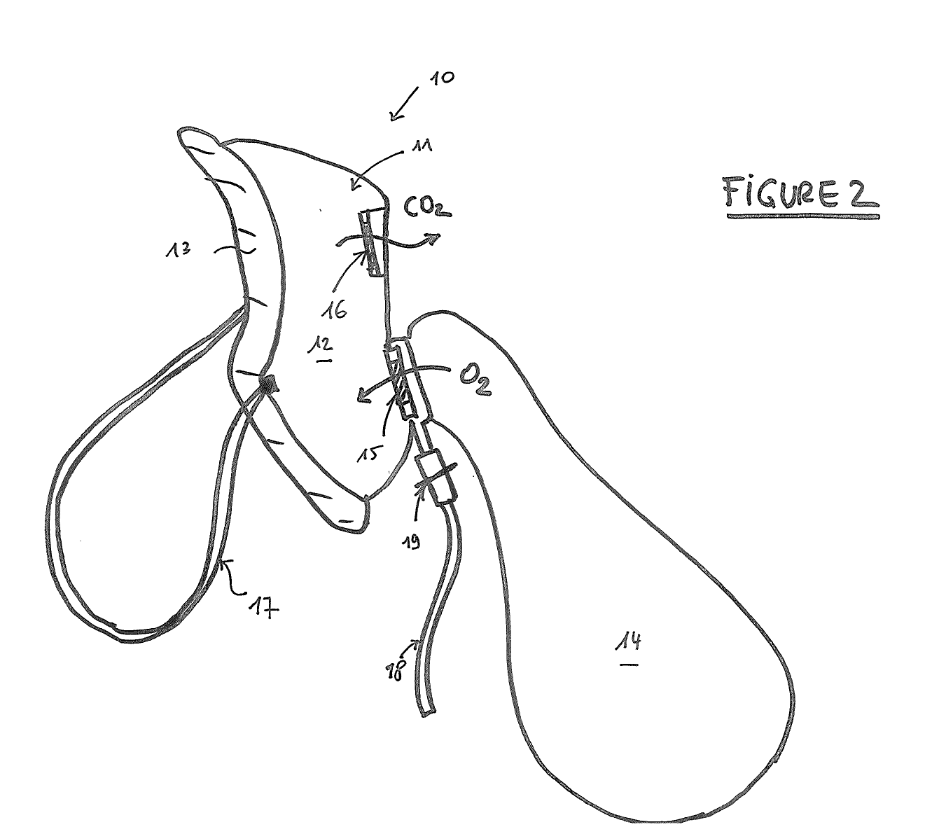

Advantageously, in the context of the present invention, a “high concentration” facial mask 10 is used to administer the gas to the patient. Such a mask 10 is represented diagrammatically in FIG. 2.

This respiratory mask 10 comprises a shell 11 forming a mask body. The shell 11 includes a gas inhalation chamber 12 which is positioned on the nasal and buccal regions of the patient, when the latter is wearing the mask 10. The airtightness is provided by an edge formed from a flexible cushion 13, for example made of silicone, which moulds to the contours of the facial regions of the patient, in particular the bridge of the nose, the regions of the cheeks located on either side of the nose, and the region under the mouth, i.e. at the level of the patient's chin.

The inhalation chamber 12 is in fluidic communication with a flexible gas reservoir 14 formed from a flexible pouch typically made of polymer (volume approximately 0.5 litre) which is filled with oxygen produced by the oxygen concentrator.

A non-return valve 15 placed between the flexible reservoir 14 and the inhalation chamber 12 makes it possible to prevent the CO₂ breathed out by the patient from mixing with the oxygen contained in the flexible reservoir 14. The CO₂ breathed out is evacuated from the inhalation chamber 12 via an expiratory valve 16 placed in the body of the mask 10.

The mask 10 is held in position on the patient's face by means of a flexible strap or of an elastic band 17.

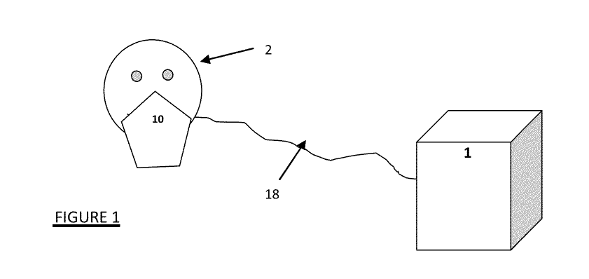

The flexible reservoir 14 is filled with the oxygen stream produced by the oxygen concentrator 1 (cf. FIG. 1) via the flexible tube 18 which connects to the mask 10 via an appropriate connector 19.

This type of facial mask 10 makes it possible to administer higher concentrations of oxygen to patients than the conventional respiratory masks, i.e. without a flexible reservoir, or higher oxygen flow rates at equal concentration of the sort to increase the patient's FiO₂, in the context of a high flow rate, i.e. 9 l/min for example.

The duration of inhalation of the gaseous composition is chosen, case by case, so as to result in a decrease and preferably in total disappearance of the migraine pain in the patient under consideration. Typically, it is at least 15 minutes to 3 hours. The administration flow rate is, for its part, about 1 l/min to 30 l/min, typically about 9 to 11 l/min.

### EXAMPLE

In order to verify the efficacy of the gaseous composition, i.e. of the gaseous medicament, according to the invention, an inhalable gaseous composition according to the invention was administered to a patient who had been suffering from chronic migraines for years, namely a 40-year-old woman.

The gaseous composition administered consisted of an oxygen-rich gaseous stream produced directly at the patient's home, i.e. in situ, by an oxygen-producing device of oxygen concentrator type operating via a cycle of PSA type.

In the present case, the oxygen concentrator having the brand name Platinum 9™ from the company Invacare™ was used, which fed a high-concentration respiratory mask from the company Intersurgical™, of the type of that represented in FIG. 2, with an oxygen-rich stream.

This concentrator makes it possible to produce an oxygen-enriched gas stream at a flow rate ranging from 3 to 9 l/min and at an oxygen concentration of 87% to 93 vol. % of oxygen, depending on the flow rate selected.

In the context of the present example, the administration of the oxygen stream produced in situ is carried out at the time of the migraine attacks, for a period of 30 minutes and at a flow rate of 9 l/min.

The results obtained resulted in a relief of the attacks after 8 to 10 minutes in the patient thus treated.

This demonstrates the efficacy of the gaseous composition according to the invention in the treatment of chronic migraines when said gaseous composition is produced directly on site, i.e. directly at the patient's home and just before the inhalation thereof.

What is claimed is:

1. A method of minimizing the episodic length of migraine headaches for patients suffering from chronic migraine episodes, the method comprising the steps of:
a) installing at one or more locations an oxygen-producing device designed so as to produce a gas stream containing more than 50% of oxygen from air for use by a patient suffering from chronic migraine episodes,
b) forming the gaseous stream containing more than 50% of oxygen from air, and
c) administering the gas stream containing more than 50% of oxygen by inhalation to the patient at or as soon as possible after the start of a migraine episode until the migraine episode at least partially subsides.

2. The method of claim 1 wherein the gas stream contains a proportion by volume of oxygen greater than 60%.

3. The method of claim 1 wherein the gas stream contains a proportion of oxygen greater than 70%.

4. The method of claim 1 wherein the gas stream contains a proportion of oxygen greater than 80%.

5. The method of claim 1 wherein the gas stream contains a proportion of oxygen less than 100%.

6. The method of claim 1 wherein the gas stream contains a proportion of oxygen less than 96%.

7. The method of claim 1 wherein the gas stream is obtained by a step of separation of oxygen from other components of air by at least one adsorbent material arranged in the oxygen-producing device.

8. The method of claim 7 wherein the at least one adsorbent material comprises a zeolite which has been at least partially exchanged with lithium and/or calcium cations.

9. The method of claim 1 wherein the oxygen-producing device is installed is at the patient's home, and the gaseous stream is formed from ambient air.

10. The method of claim 1 wherein the gas stream is administered by inhalation via a respiratory mask comprising a mask body comprising an inhalation chamber in fluidic communication with a flexible reservoir containing at least one part of the gaseous stream.

11. The method of claim 10, wherein the respiratory mask further comprises a non-return expiratory valve placed between the flexible reservoir and the inhalation chamber adapted to evacuate the exhalation gases of a patient to prevent exhalation gases breathed out by the patient from mixing with and diluting the oxygen concentration in the gaseous stream contained in the flexible reservoir.
